(19) United States
(12) Patent Application Publication
(10) Pub. No.: US 2010/0292632 A1
(43) Pub. Date: Nov. 18, 2010

(54) Transdermal Micro-Patch

(76) Inventors: Maureen L. Mulvihill, Bellefonte, PA (US); Brian M. Park, State College, PA (US); Gareth Knowles, Williamsport, PA (US)

Correspondence Address: Law Offices of Michael Crilly, 104 South York Road, Hatboro, PA, 19040, US

(21) Appl. No.: 12/812,799

(22) PCT Filed: Feb. 13, 2009
(86) PCT No.: PCT/US09/34038

§ 371 Date: Jul. 14, 2010

Related U.S. Application Data
(60) Provisional application No. 61/065,850, filed on Feb. 15, 2008.

(51) Int. Cl. A61N 7/00; A61M 35/00

(52) U.S. Cl. 604/22

(57) ABSTRACT

A transdermal micro-patch for use with living tissue is provided. The micro-patch includes a first membrane, a reservoir, a micro-pump, flextensional transducers, a microelectronics circuit, and an optional sensor. The first membrane is permeable to allow the passage of fluid in either a unidirectional or bidirectional fashion. The reservoir is a container-like element capable of storing a fluid removed from or communicated into the tissue. The micro-pump facilitates transport of the fluid between the reservoir and first membrane. The flextensional transducers generate ultrasonic waves which are separately communicated into the tissue to transport fluid between the first membrane and tissue. Ultrasonic waves could interact to enhance the performance of the micro-patch. The microelectronics circuit controls both flextensional transducers and the micro-pump. The sensor could be embedded within the micro-patch to monitor temperature, pressure, or flow rate so as to avoid damage or irritation to the tissue.

### CROSS REFERENCE TO RELATED APPLICATIONS

This application is based upon and claims priority from Patent Cooperation Treaty Application No. PCT/US2009/034038 filed Feb. 13, 2009 and U.S. Provisional Application No. 61/065,850 filed Feb. 15, 2008, both entitled Transdermal Micro-Patch, the contents of which are hereby incorporated in their entirety by reference thereto.

### FEDERALLY SPONSORED RESEARCH AND DEVELOPMENT

None.

### BACKGROUND OF THE INVENTION

1. Field of the Invention

The invention generally relates to a fully-functional, self-contained, needle-free system for the administration of fluids, example including medications, oxygen, and nutrients, into tissues or wounds and for the extraction of such fluids through skin. Specifically, the invention is a compliant transdermal patch including first and second membranes disposed about one or more separately functional flextensional transducers, a micro-pump attached to a reservoir, an optional encapsulating matrix, an optional feedback sensor, and a microelectronics circuit which controls function of the micro-pump and transducers allowing for the delivery or extraction of a fluid or the like through the first membrane.

2. Background

The effective treatment of injuries, diseases, and other medical related conditions remain a challenge for medical practitioners. Skin sores, burns, bedsores, and open wounds are particularly challenging. Many open wounds do not respond to present treatment practices and never properly heal. In many instances, the circulatory system within and adjacent to a wound is compromised, thus preventing oxygen from reaching the affected tissues. This lack of oxygen, or prolonged period of oxygen deprivation, is commonly referred to as hypoxia and can slow or stop the natural healing process. Quite often the result is permanent, irreversible damage to tissues within and adjacent to a wound, which sometimes leads to the loss of a limb, horrific scarring or disfigurement, and/or death.

Any increase in the amount of oxygen to a wound site, particularly an increase in the subcutaneous partial pressure of oxygen, can improve healing and bacterial defenses. The related arts include a variety of devices and methods capable of delivering oxygen to a wound site otherwise deprived of oxygen. For example, topical colloidal dressings are disclosed by Artandi in U.S. Pat. No. 3,157,524 entitled Preparation of Collagen Sponge and by Berg et al. in U.S. Pat. No. 4,320,201 entitled Method for Making Collagen Sponge for Medical and Cosmetic Uses. The application of super-oxygenated compositions is disclosed by McGrath et al. in U.S. patent application Ser. No. 10/637,205 entitled Method for Increasing Tissue Oxygenation. The administration of oxygen to a patient generally in a hyperbaric chamber or to a specific location by “topical hyperbaric” methods are disclosed by Loori in U.S. Pat. No. 4,801,291 entitled Portable Topical Hyperbaric Apparatus. While known devices and methods are capable of oxygenating a trauma site, they are bulky, time and labor intensive, diffusion based, and unable to effectively deliver oxygen to hypoxic tissues. Furthermore, some topical hyperbaric treatments, which administer a peroxide solution, produce free radicals causing further damage to tissues within the treatment area.

Transdermal delivery devices and methods employing an ultrasonic transducer to deliver drugs and medication therapies are known within the art. In general, the ultrasound transducer transforms an electrical signal into an acoustic vibration causing the skin to be more permeable, thus enabling the delivery of a fluid into the blood system or the extraction of an interstitial fluid. Specific examples include glucose monitoring and insulin delivery via a sonicator. However, these conventional transdermal delivery and extraction devices are too large for portable patch-type systems. Furthermore, conventional ultrasonic-based transdermal systems are known to damage tissues within the treatment zone, thus resulting in the loss of hair follicles, destruction of sebaceous glands, and necrosis of cutaneous musculature.

Conventional transducer technologies consisting of single and layered assemblies of a piezoelectric ceramic are hindered by the maximum strain limit of such materials. For example, the maximum strain limit of conventional piezoelectric ceramics is about 0.1% for polycrystalline materials, such as ceramic lead zirconate titanate (PZT), and 0.5% for single crystal materials. Accordingly, a large number of piezoelectric ceramic elements in a stacked arrangement are required to achieve useful displacement or actuation to produce ultrasonic waves. In terms of an ultrasonic transdermal patch, piezoelectric ceramics preclude the implementation of portable and convenient micro-patches.

As is readily apparent from the discussions above, the related arts do not include a compact transdermal patch allowing for the efficient and effective delivery of a fluid into or extraction of a fluid from living tissue while also avoiding damage and irritation to such tissues.

Therefore, what is required is a self-contained, fully-function transdermal patch capable of delivering a nutrient to and/or extracting a fluid from tissues while minimizing the extent and degree of trauma and irritation experienced by tissues immediately adjacent to the patch.

### SUMMARY OF THE INVENTION

An object of the present invention is to provide a self-contained, fully-function transdermal patch capable of delivering a nutrient to and/or extracting a fluid from tissues while minimizing the extent and degree of trauma and irritation experienced by tissues immediately adjacent to the patch.

The compliant, transdermal micro-patch includes at least one flextensional transducers, a micro-pump attached to a reservoir, a first membrane, a second membrane, and a microelectronics circuit electrically communicating with the transducers and micro-pump. Flextensional transducers, micro-pump, reservoir, and microelectronics circuit are disposed along the first membrane and could be sealed between the first and second membranes, with the flextensional transducers further sealed and suspended within an optional encapsulating matrix composed of a high impact polyurethane resin. The flextensional transducers and a conduit from the micro-pump contact the interior surface of the first membrane. The micro-pump communicates fluid between the reservoir and the first membrane. The transducers independently generate ultrasonic waves which are separately communicated into living tissue, thereby increasing the permeability of the tissue so as to transport fluid between the tissue and the first membrane. The microelectronics circuit controls operability of the micro-pump and transducers for the effective delivery/removal of a fluid or the like between the reservoir and the first membrane via the micro-pump and conduits.

In some embodiments, an adhesive is disposed along the first membrane opposite of the transducers to facilitate attachment of the micro-patch to skin.

In yet other embodiments, the first membrane is preferred to allow one-way or two-way flow of a fluid out from or into the micro-patch.

In still other embodiments, the micro-patch could include a sensor to determine one or more conditions within the micro-patch or tissues contacting the micro-patch indicative of damage or irritation.

In still yet other embodiments, the transducers could communicate at least two separate waves into the living tissue so as to interact along at least one region, thereby increasing the permeability of such tissues without irritation or damage thereto.

The flextensional transducers could include a piezoelectric ceramic driving cell disposed within a frame, platen, housing, end-caps or other geometry which amplifies the transverse, axial, radial or longitudinal motions or strains of the driving cell in one direction to obtain larger displacement in a second or a preferred direction, than otherwise achievable with the piezoelectric ceramic alone. The acoustic vibrations achievable with flextensional transducers could increase skin permeability and the efficiency with which oxygen is delivered to a treatment area while minimizing irritation or damage to the delivery site. Flextensional transducers are compact and thereby compatible within micro-patch devices.

Cymbal-shaped flextensional transducers, like those described by Newnham et al. in U.S. Pat. No. 5,729,077 entitled Metal-Electroactive Ceramic Composite Transducer, use metal end-caps to enhance the mechanical response of a piezoceramic disk to an electrical input. In a typical cymbal transducer, high frequency radial motion within a disk composed of a piezoelectric ceramic is transformed into low frequency (20-50 kHz) displacement motion through a cap-covered cavity. A cymbal transducer takes advantage of the combined expansion in the piezoelectric charge coefficient d₃₃, representing induced strain in direction 3 per unit field applied in direction 3, and contraction in the d₃₁, representing induced strain in direction 1 per unit field applied in direction 3, by a piezoelectric ceramic, along with the flextensional displacement of the metal end-caps. The end-caps about the ceramic disk enable both longitudinal and transverse responses to contribute to the strain in the desired direction, creating an effective piezoelectric charge constant (d_{eff}) according to the equation

d_{eff}=d₃₃+(−A*d₃₁)

where A is the amplification factor of the transducer which can be as high as 100.

End-cap materials could include, but are not limited to, brass, steel, and Kovar™, a registered trademark of CRS Holdings, Inc. of Wilmington, Del. Metal end-caps also provide additional mechanical stability, ensuring a longer effective lifetime for the transducer. End-caps could include a variety of profiles and shapes.

Flextensional transducers could be electrically activated in a sequenced arrangement so as to produce low-level ultrasonic waves which open micro-channels within the stratum corneum, allowing an oxygen-rich fluid communicated from the patch to reach damaged and hypoxic tissues or allowing fluids within tissues to be extracted therefrom. Low-level ultrasonic waves, typically in the range of 20 kilohertz (kHz), minimize damage and other changes within the treatment area.

Micro-channels are formed within the living tissue as the ultrasonic waves traverse and cavitate the tissue. Cavitation includes the rapid expansion and collapse of gaseous bubbles in response to an alternating pressure field and broadly includes stable and transient modes. Stable cavitations occur when a cavity oscillates about its equilibrium radius in response to relatively low acoustic pressures. Transient cavitations occur when the equilibrium bubble radius greatly varies within a few acoustic cycles. During transient cavitations, bubbles rapidly and violently collapse because of high acoustic pressures and localized elevated temperatures. The violent hydrodynamic forces associated with a collapsing bubble can severely damage biological tissues and release free radicals. Ultrasound in the megahertz (MHz) range also produces cavitation, although much higher pressures are required to exceed the cavitation threshold associated with cell disruption and damage tissue. The invention described herein minimizes transient cavitations in order to avoid the disruption of cells and damage to tissues contacting the micro-patch.

Several advantages are offered by the invention. The invention facilitates the needle-free, automated and safe delivery of nutrients and other fluids required to treat open wounds. The invention minimizes the risk of infection otherwise caused by needles. The invention eliminates the need for manual fluid pressure for aspiration or irrigation by automation via a micro-pump. The invention facilitates continuous use or reuse via a refillable reservoir. The invention facilitates continuous transfer or extraction of a large amount of fluid using the micro-pump assembly in a fashion that enables continuous usage or refill/drain without removing the transdermal assembly from the patient. The invention can be integrally manufactured, including lightweight and compact power electronics and control mechanisms, so as to have a small footprint to minimize the tissue area affected by the device and to minimize discomfort to the wearer, thus providing a compact, wearable solution. The invention offers a wide range of power solutions, including propane or hydrogen fuel cells, batteries, and DC power via a wall outlet. The invention is readily adaptable to a variety of computers via an interface to monitor and control the reservoir, flow from the reservoir, and flow into the user.

The above and other objectives, features, and advantages of the preferred embodiments of the invention will become apparent from the following description read in connection with the accompanying drawings, in which like referenced numerals designate the same or similar elements.

### BRIEF DESCRIPTION OF THE DRAWINGS

Additional aspects, features, and advantages of the invention will be understood and will become more readily apparent when the invention is considered in the light of the following description made in conjunction with the accompanying drawings.

FIG. 1 is a partial section view illustrating a transdermal micro-patch including a pair of cymbal-shaped transducers, a micro-pump, a reservoir, a matrix, and a microelectronics circuit disposed between a flexible, porous first membrane and a flexible second member and further contacting living tissue in accordance with one embodiment of the invention.

FIG. 2 is a partial section view illustrating attachment of the transdermal micro-patch shown in FIG. 1 onto living tissue and the separate communication of ultrasonic waves into the tissue by the transducers which interact within the tissue in accordance with one embodiment of the invention.

FIG. 3a is a cross section view illustrating a two-by-two arrangement of flextensional transducers within a generally square-shaped micro-patch in accordance with one embodiment of the invention.

FIG. 3b is a cross section view illustrating a three-by-three arrangement of flextensional transducers within a generally square-shaped micro-patch in accordance with one embodiment of the invention.

FIG. 3c is a cross section view illustrating a pair of flextensional transducers within a generally rectangular-shaped micro-patch in accordance with one embodiment of the invention.

FIG. 3d is a cross section view illustrating the arrangement of five flextensional transducers within a generally circular-shaped micro-patch in accordance with one embodiment of the invention.

FIG. 4 is a cross section view illustrating electrical connectivity within a two-by-two arrangement of flextensional transducers comprising a micro-patch in accordance with one embodiment of the invention.

FIG. 5 is a block diagram illustrating high-level functional aspects of control circuitry attached to flextensional transducers in accordance with one embodiment of the invention.

FIG. 6 is a block diagram illustrating electrical connectivity between a micro-patch and an amplifier, a signal generator, and a power supply in accordance with one embodiment of the invention.

FIG. 7 is a schematic diagram illustrating components and architecture within a microelectronics circuit in accordance with one embodiment of the invention.

### DETAILED DESCRIPTION OF THE INVENTION

Reference will now be made in detail to several preferred embodiments of the invention that are illustrated in the accompanying drawings. Wherever possible, same or similar reference numerals are used in the drawings and the description to refer to the same or like parts or steps. The drawings are in simplified form and are not to precise scale. The words communicate, connect, couple, link, and similar terms with their inflectional morphemes do not necessarily denote direct and immediate connections, but also include connections through intermediary elements or devices.

For purposes of this invention, fluid is understood to broadly include non-biological and biological substances in liquid and/or gaseous form with or without solid particulates.

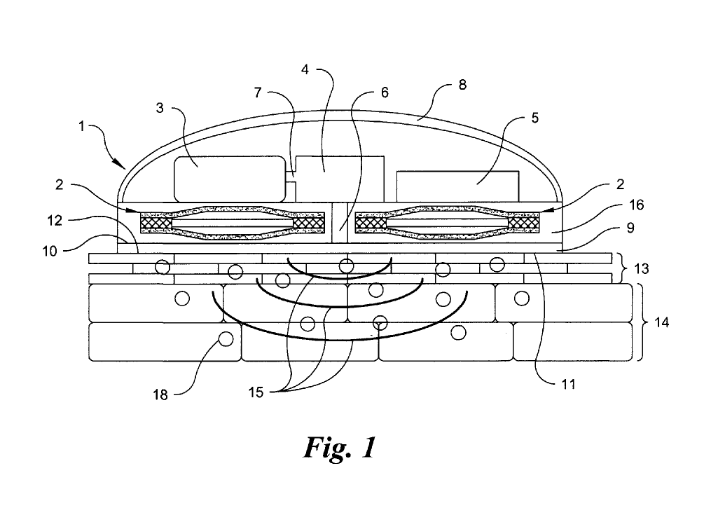

Referring now to FIG. 1, the transdermal micro-patch 1 could include one or more flextensional transducers 2, a micro-pump 4, a reservoir 3, and a microelectronics circuit 5 disposed between a first membrane 9 and an optional second membrane 8 so as to form a generally compliant device of substantially planar extent. The flextensional transducers 2, micro-pump 4, reservoir 3, and microelectronics circuit 5 are either rigid, semi-rigid, or flexible elements which could be bonded to the first membrane 9 via an adhesive in an arrangement which maximizes the flexibility or pliability of the transdermal micro-patch 1. In some embodiments, one or more components could be encapsulated within a matrix 16, composed of a flexible or pliable polymer, elastomer, or the like, via known methods, including, but not limited to, injection molding and gravity pour casting with or without vacuum assist. In yet other embodiments, components are separately bonded to the first membrane 9 or the second membrane 8 or both, and thereafter encapsulated between the first and second membranes 9, 8 which are attached via an adhesive or ultrasonic weld about the perimeter of the transdermal micro-patch 1. In other embodiments, the flextensional transducers 2, micro-pump 4, reservoir 3, and microelectronics circuit 5 could be attached to the interior surface 10 via an epoxy so as to minimize stiffening along the otherwise compliant first membrane 9.

The first membrane 9 is a flexible or pliable material of generally planar extent capable of contacting skin and other living tissue without irritation. The first membrane 9 is preferred to be composed of a material which is porous, permeable, open celled, or woven so as to allow a fluid to pass into and through the first membrane 9 in either a bi-directional or unidirectional fashion. One such exemplary material includes, but is not limited to, ethylene vinyl acetate sold under the trademark CoTran™ by the 3M™ Company. In some embodiments, the first membrane 9 could function similar to a sponge so as to temporally hold or store fluid before transport into or out of the transdermal micro-patch 1.

The second membrane 8 is likewise of generally planar extent and capable of contacting skin and other living without irritation. It is preferred for the second membrane 8 to be composed of a medical grade non-porous polymer or elastomer composition which is flexible or pliable, one example being polypropylene.

The flextensional transducers 2 are piezoelectric elements capable of generating ultrasonic waves 15 which transverse the epidermis 13 and dermis 14, or other living tissues, in contact with the transdermal micro-patch 1. The flextensional transducer 2 are preferred to be disk-shaped or cymbal-shaped elements, like those described by Newnham et al. in U.S. Pat. No. 5,729,077 entitled Metal-Electroactive Ceramic Composite Transducer, which is incorporated in its entirety herein by reference thereto. While cymbal-shaped transducers are disclosed, other flextensional transducers are possible, such as those having a square or rectangular cross-section along a plane perpendicular to the amplification axis. Yet other flextensional-type transducers could include thin-layer laminate structures like those described by Knowles et al. in U.S. Pat. No. 6,665,917 entitled Method of Fabricating a Planar Pre-stressed Bimorph Actuator.

The flextensional transducers 2 are positioned within the transdermal micro-patch 1 so as to directly or nearly directly contact the interior surface 10 of the first membrane 9 and disposed between the first membrane 9 and the reservoir 3, micro-pump 4, and microelectronics circuit 5, the latter elements generally arranged along a substantially common plane. This arrangement ensures that the ultrasonic waves 15 produced by the individual flextensional transducers 2 are communicated into and through the first membrane 9 with minimal adverse attenuation.

In some embodiments, the flextensional transducers 2 could be encapsulated within a matrix composed of an elastomeric material, urethane resin, or the like, as represented in FIG. 1. An exemplary resin is a polyurethane composition identified as URA-BOND FDA 24N manufactured by Resin Technology Group, LLC.

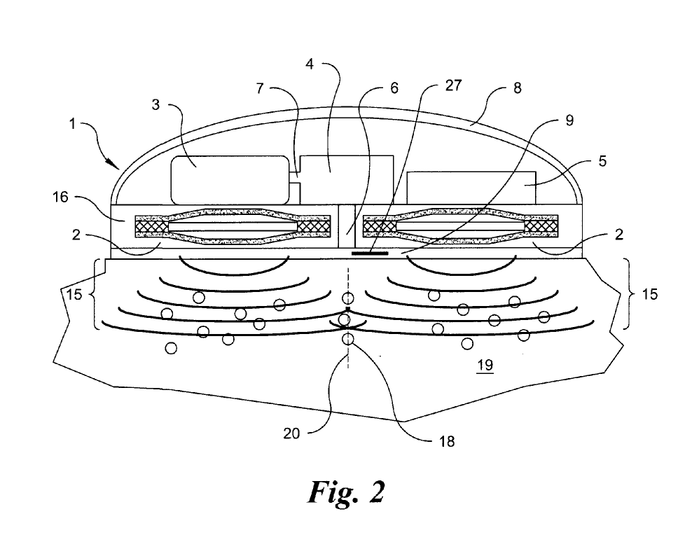

In other embodiments, the arrangement and functionality of the flextensional transducers 2 could communicate ultrasonic waves 15 which combine to form a single waveform having a simple or complex arcuate profile, a linear profile, or a combination thereof, whereby an example of the simple and complex arcuate profiles are graphically depicted in FIGS. 1 and 2, respectively.

The ultrasonic waves 15 produced by the flextensional transducers 2 are characterized as a plurality of waves which originate from the source and travel along a common direct. The energy within each ultrasonic wave 15 should be sufficient, either separately or in combination, to form micro-channels within the epidermis 13 and dermis 14 and to move or transport fluid 18 residing within either the first membrane 9 or epidermis 13 and dermis 14 in a preferred direction.

In yet other embodiments, the exterior surface 11 of the transdermal micro-patch 1 could include an adhesive 12 in a layered or thin-film arrangement. In preferred embodiments, the adhesive 12 is disposed about the periphery of the transdermal micro-patch 1 so as to prevent the leakage of fluid 18 as it passes from or to the transdermal micro-patch 1. It is also possible for the adhesive 12 when contacting tissues to form a pocket within which fluid 18 pools prior to entering or after exiting the tissue. The adhesive 12 could be a commercial-grade composition used within the medical field, preferably water resistant, and capable of securing the transdermal micro-patch 1 to the outer surface of living tissue without irritation.

The micro-pump 4 could be a commercially available mechanical or non-mechanical device, preferably piezoelectric actuated, capable of rapidly moving fluid 18 into and through small spaces at a flow rate in the range of micro-liters to milliliters per minute. In some embodiments, the micro-pump 4 could pressurize the fluid 18 stored within the reservoir 3 so that it moves into and through the first membrane 9. In other embodiments, the micro-pump 4 could create a vacuum-like condition within the first membrane 9 or a cavity within the transdermal micro-patch 1 so as to draw fluid 18 within the dermis 14 or other living tissue into the transdermal micro-patch 1, thereafter directed into the reservoir 3 for storage. An exemplary micro-pump 4 could include the device described by Junwu, K. et al. in Design and Test of a High Performance Piezoelectric Micro-Pump for Drug Delivery, Sensors and Actuators A: Physical, Vol. 121, Issue 1, Pages 156-161. Control circuitry for the micro-pump 4 could be housed within the micro-pump 4 or provided on the microelectronics circuit 5.

The reservoir 3 is a chamber or container-like element composed of a lightweight material, such as a polymer, which is capable of storing at least several milliliters of fluid 18 without leakage, contamination, or spoilage. The reservoir 3 is required to have a hole through which fluid 18 enters or leaves the reservoir 3. In preferred embodiments, the reservoir 3 should allow for the insertion of a needle for the injection or extraction of a fluid 18.

The micro-pump 4 includes tube-shaped first and second conduits 6, 7 which extend from the micro-pump so as to enable a fluid 18 to pass into and through the micro-pump 4. One end of the first conduit 6 contacts the interior surface 10 of the first membrane 9. The first conduit 6 could be secured to the first membrane 9 via a compression fit, via hose barb, or via an adhesive disposed about the perimeter at the interface between the first conduit 6 and first membrane 9. In some embodiments, it is preferred that the first conduit 6 be disposed between two or more flextensional transducer 2 so as to ensure a more uniform delivery of a fluid 18 into the surrounding tissue. One end of the second conduit 7 is fixed about a hole along the wall of the reservoir 3 via an adhesive or mechanical fastener. This arrangement allows fluid 18 to flow from the reservoir 3 through the second conduit 7, micro-pump 4, and first conduit 6 and into and through the first membrane 9 when the transdermal micro-patch 1 is employed as a delivery system. This arrangement also allows fluid 18 to flow through the first membrane 9 and into and through the first conduit 6, micro-pump 4, and second conduit 7 and into the reservoir 3 when the transdermal micro-patch 1 is employed as an extraction system.

In some embodiments, the micro-pump 4 could include a removable cartridge that facilitates the continuous transdermal fluidic delivery or extraction of a fluid 18 without adjustment, removal, or reconfiguration of the reservoir 3, micro-pump 4, flextensional transducers 2, first and second membranes 9, 8, and/or microelectronics circuit 5. The transdermal micro-patch 1 could be attached to the tissue 19 so that the first membrane 9 and/or second membrane 8 act as a barrier until the transdermal fluidic transfer is safe to continue.

The microelectronics circuit 5 is electrically connected to the flextensional transducers 2 and micro-pump 4 and could include control circuitry and a power supply capable of driving one or more flextensional transducers 2. AC-powered drive electronics would need to generate a frequency output at 10 to 100 kHz, preferably from 20 to 30 kHz, and most preferably at 28 kHz to provide an intensity from 0.01 to 0.1 W/cm².

The microelectronics circuit 5 includes both hardware and software required to control the functionality of the micro-pump 4 and flextensional transducers 2. Circuitry is disposed on a rigid or semi-rigid substrate commonly used with printed circuit boards (PCB). Circuitry could include a compact drive electronics section, a microcontroller unit (MCU), and an interface port facilitating control via an external controller. In preferred embodiments, the drive electronics are electrically connected to the flextensional transducers 2. Exemplary microelectronics circuits 5 could include devices sold by Altium, Inc. located in Carlsbad, Calif.

Control circuits could include a number of operationally orientated programs for operating the micro-pump 4 and flextensional transducers 2. Programs could further include a power management feature that allows the transdermal micro-patch 1 to operate for an extended period of time without an external power supply or in a mode which optimizes delivery or extraction characteristics achieved by the micro-pump 4 and flextensional transducers 2 based on flow conditions or other conditions measured within the transdermal micro-patch 1 and/or tissue immediately adjacent thereto. Conservation software could include a low standby current design for use when the transdermal micro-patch 1 is neither delivering nor extracting a fluid 18.

In some embodiments, the MCU could control the general operation of the transdermal micro-patch 1 under at least some element of software of firmware control. The MCU could operate the micro-pump 4 and flextensional transducer 2 so that either one or both devices are functioning at any given time. In one example, it is possible for some or all of the flextensional transducers 2 to function while the micro-pump 4 is idle such as when the device is first activated and preparing the delivery/extraction site. In another example, it is possible for the micro-pump 4 to function when the flextensional transducers 2 are idle such as when conditions within tissue adjacent to the transdermal micro-patch 1 have been optimized for the delivery or extraction of a fluid 18 or when conditions suggest damage or irritation to the tissue. In yet another example, it is possible for the micro-pump 4 and flextensional transducers 2 to operate simultaneously with adjustments to the flow rate via adjustments to the operational speed of the micro-pump 4 and/or the intensity, frequency, displacement, and/or phasing of the flextensional transducers 2.

Referring now to FIG. 2, the transdermal micro-patch 1 could include two or more flextensional transducers 2 which are activated simultaneously or in a phased arrangement so that the resultant ultrasonic waves 15 interact or collide along one or more interaction zones 20 within the tissue 19. In some embodiments, a higher absorption rate and/or deeper absorption depth could be beneficial to enhance the volume of fluid 18 delivered to the tissue 19, to increase the total volume of tissue 19 exposed to the fluid 18, or to ensure the delivery or extraction of fluid 18 from tissues or internal organs beyond the dermis 14.

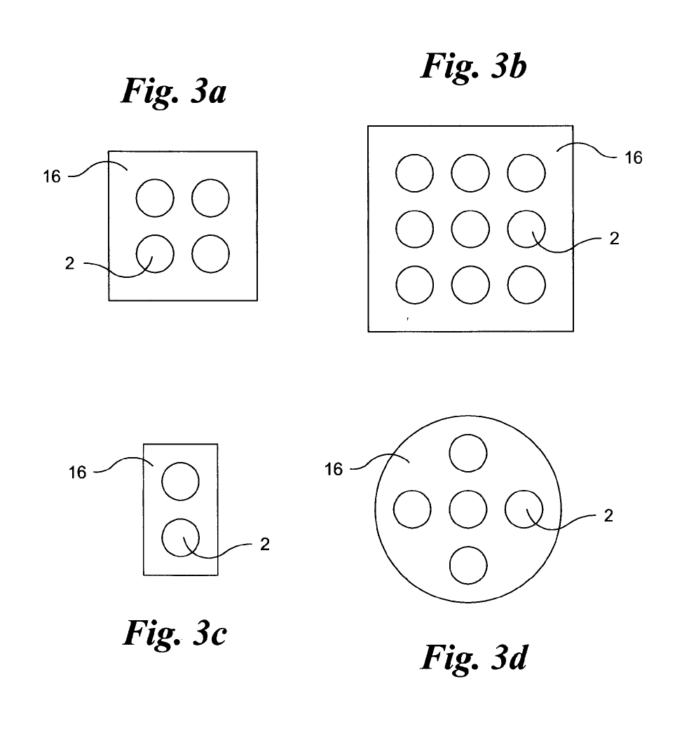

The flextensional transducers 2 could be arranged in a variety of symmetric or asymmetric patterns within one or more planes relative to the first membrane 9 and about the micro-pump 4. For example, FIGS. 3a and 3b show a matrix 16 having a two-by-two and three-by-three arrangement of low-profile flextensional transducers 2, respectively, within a square-shaped transdermal micro-patch 1. In another example, FIG. 3c shows a matrix 16 including a two-by-one arrangement of low-profile flextensional transducers 2 within a rectangular-shaped transdermal micro-patch 1. In yet another example, FIG. 3d shows a matrix having five flextensional transducers 2 symmetrically arranged within a circular-shaped transdermal micro-patch 1.

Referring again to FIGS. 3a-3d, the flextensional transducers 2 are electrically activated by the microelectronics circuit 5 to achieve a variety of operational modes. In one example, all flextensional transducers 2 could be activated simultaneously via one or more inputs signals so as to achieve one or more mechanical responses. In another example, the flextensional transducer 2 could be activated via one or more input signals which are phase shifted, time shifted, sequenced, and/or otherwise differ in frequency and/or voltage. The mechanical response of the flextensional transducer 2 could be used separately or in combination to tailor the number, size, and shape of the ultrasonic waves 15 or the interaction zones 20 formed thereby within the delivery/extraction site.

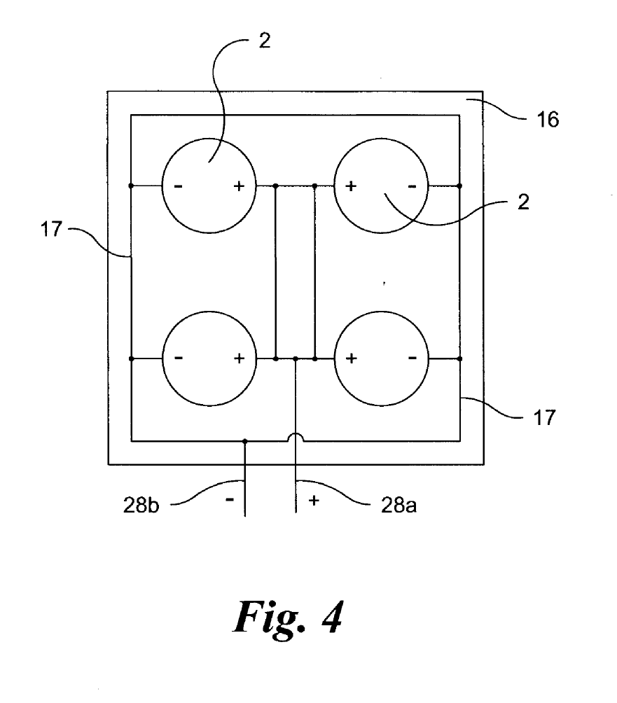

Referring now to FIG. 4, flextensional transducers 2 disposed within a common matrix 16 are electrically coupled to each other and to either an external or internal power supply via conductive wires 17. Each flextensional transducer 2 is poled so as to include a pole of positive polarity and a pole of negative polarity, identified by the symbols “+” and “−” in FIG. 4, respectively. Flextensional transducers 2 could be electrically connected so that all positive poles are coupled to one conductive lead 28a and all negative poles are coupled to another conductive wire 28b. Thereafter, the conductive leads 28a, 28b are electrically coupled directly to a power supply and/or to the microelectronics circuit 5. Other electrically connectivity arrangements are possible.

In some embodiments, it could be advantageous to include a sensor 27 capable of quantifying the magnitude of events, either in absolute or relative terms, within the transdermal micro-patch 1 and/or the tissue 19 adjacent thereto. Referring again to FIG. 2, a sensor 27 is generally represented to reside within the first membrane 9, although it is likewise possible for the sensor 28 to be disposed along the exterior surface 10 of the first membrane 9 or within the matrix 16 or other location which minimizes the filtering or attenuation effects by the transdermal micro-patch 1 and/or components thereof. The sensor 27 could measure the flow rate of fluid 18 into or out of the transdermal micro-device 1 or the temperature, pressure, or frequency and amplitude of vibrations within the transdermal micro-device 1 and/or the tissue 19. In preferred embodiments, the sensor 27 could be a thin-film, fine-wire, or low-profile thermocouple, accelerometer, flow meter, or pressure transducer, capable of rapidly measuring the respective parameter within the delivery/extraction zone. In other embodiments, the sensor 27 could quantify conditions that directly or indirectly correlate to cavitation events produced within the tissue 19 by the flextensional transducers 2. The sensor 27 could be electrically coupled to the microelectronics circuit 5 which would actively monitor measured data so as to implement adjustments to the micro-pump 4 and/or flextensional transducer 2 as appropriate to avoid damage and/or irritation to the tissue 19 or to optimize delivery or extraction of a fluid 18.

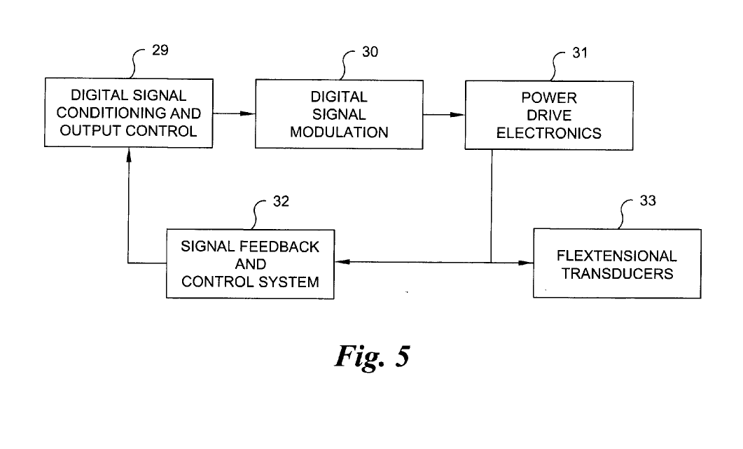

Referring now to FIGS. 5-7, various electronic components and architecture applicable to an exemplary transdermal micro-patch 1 are shown. Diagram are not meant to be exhaustive of the electrical components, connections, and architecture used within the transdermal micro-patch 1, but are merely illustrative to assist in describing the methods and hardware utilized to operate the device in the manner described herein. There may be additional processors, PROM, RAM or ROM memory devices or both including NAND/NOR flash-type memory, masked ROM, or a hard drive, or any other storage medium for storing and executing control and operation information.

Referring again to FIG. 5, the methodology of the control circuitry could include a conditioning/control step 29, a modulation step 30, and a power electronics step 31. The power electronics step 31 communicates directly or indirectly with the flextensional transducers 33. An optional feedback/control step 32 could be electrically coupled to the flextensional transducers 33 via a bidirectional arrangement and electrically coupled to the signal/output control 29 via a unidirectional arrangement.

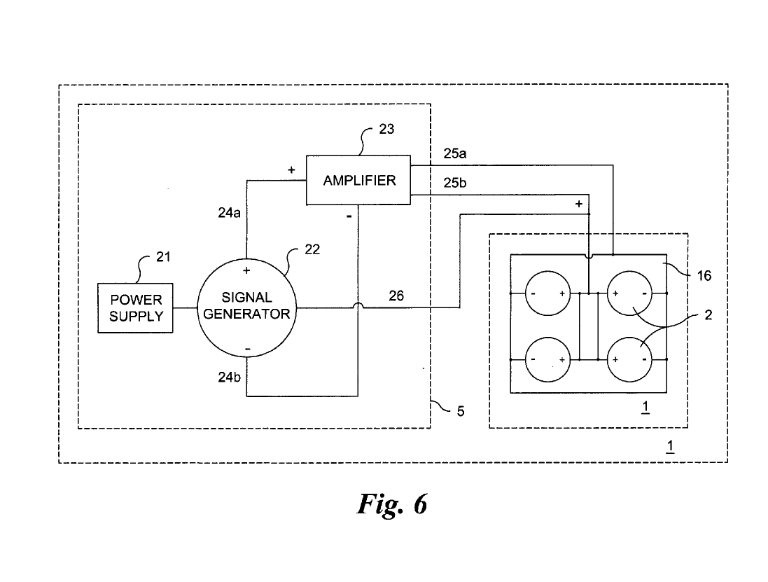

Referring again to FIG. 6, a matrix 16 is shown including four flextensional transducers 2 which are electrically coupled to an amplifier 23 via output leads 25a, 25b. Thereafter, the amplifier 23 is electrically coupled to a signature generator 22 via input leads 24a, 24b. The signature generator 22 is electrically coupled to a power supply 21 and could include an optional phase feedback 26 electrically coupled to an output lead 25a. Power supply 21, signal generator 22, amplifier 23, and feedback 26 elements include commercially available components.

The power supply 21 could include elements which provide a readily available source of DC power, one non-limiting example being batteries, or AC power, one non-limiting example being a power cord attached to an outlet. In some embodiments, the batteries could be housed within the transdermal micro-patch 1 in a non-removable fashion requiring the replacement of the patch when the power supply 21 is depleted. In other embodiments, the transdermal micro-patch 1 could include a removable panel disposed along the second membrane 8 allowing access to the power supply 21. In yet other embodiments, the transdermal micro-patch 1 could include leads which facilitate connection to an external power supply.

The signal generator 22 could include one or more channels which communicate a voltage waveform to the amplifier 23. Waveforms could include, but are not limited to, sine, square, triangular, and sawtooth signals. The signal generator 22 could shift the voltage waveforms in time or phase to achieve the desired mechanical response by each flextensional transducer 2. The amplifier 23 further adjusts the amplitude of the waveform communicated to the flextensional transducers 2 to further refine the mechanical response. The phase feedback 26 also enables the signal generator to refine the input waveform in real-time. In some embodiments, the refinement process could also consider conditions monitored by the sensor 27 described herein.

In some embodiments, the flextensional transducers 2 could be separately packaged from the power supply 21, signal generator 22, and amplifier 23 so that electrical coupling between control elements and the transdermal micro-patch 1 is via the output leads 25a, 25b. In other embodiments, the power supply 21, signal generator 22, and amplifier 23 could reside within the microelectronics circuit 5 housed within the transdermal micro-patch 1 or as a separate element therefrom.

The ON and OFF functionality of the transdermal micro-patch 1 could be controlled via various means. In some embodiments, the transdermal micro-patch 1 could include a depression-type switch disposed along the second membrane 8. In other embodiments, the transdermal micro-patch 1 could be operable via a switch attached to a control module, separate and apart from the patch, including the power supply 21, signal generator 22, and amplifier 23 described herein. In yet other embodiments, a pair of low-profile batteries could be housed within the transdermal micro-patch 1, but electrically isolated from the control circuitry via a removable, non-conductive strip. The strip is manually removed by the user so as to allow electrical contact between the power source and circuitry within the patch, thereby energizing the control circuit. Other control arrangements including switch or switch-like arrangements or their equivalents are possible.

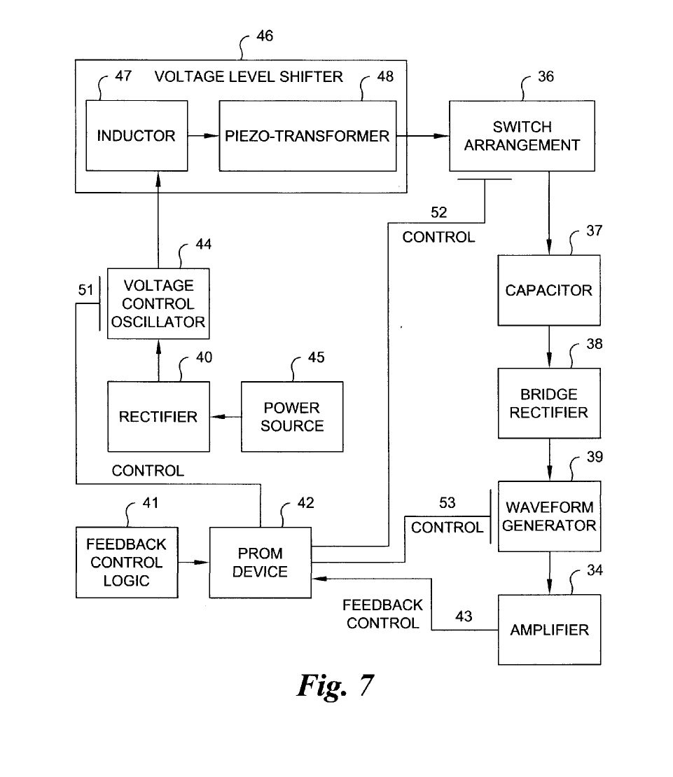

Referring again to FIG. 7, the microelectronics circuit 5 could include a power source 45, as described herein. An AC source could be rectified via a simple rectifier 40; although, in many applications the output need not be particularly well regulated or with low noise, allowing the otherwise optional rectifier 40 to be a simple bridge network.

After the rectifier 40, the DC voltage is then communicated to a voltage control oscillator 44 that yields a pulsed, sinusoidal, square, or other waveform which is communicated to a voltage level shifter 46. In preferred embodiments, the voltage control oscillator 44 is implemented as a digital encoder on a PROM device that can simultaneously incorporate feedback control logic 41, whose input is sensed outputs taken at the electrical load of the flextensional actuators 2. A stand alone device is likewise applicable. The output could consist of a pulse train communicated to the drive or input side of a piezo-transformer 48. Such devices are capacitive in nature, with no resistance to speak of; therefore, negligible loss is incurred. However, since such devices are capacitive dominated, it is problematic to directly pump a non-sinusoidal waveform into the piezo-transformer 48 as it will want to pull significant current. Therefore, a small inductor 47 could be included on the input side of the piezo-transformer 48.

In some embodiments, the piezo-transformer 48 should communicate a voltage which is greater than the required voltage level. For example, the piezo-transformer 48 might output a voltage of 300V for a flextensional transducer 2 requiring a drive voltage of 200V. From a functional standpoint, a ceramic transformer output voltage selection point is set for all or each flextensional transducer 2. Each time the output waveform crosses the selected value, a comparator logic controlled switch turns ON so as to enable current to flow at that voltage set level. This process takes a portion of the output waveform at each pass of the threshold value at a very high repeat rate. For example, the repeat rate could be 10 μs for a 100 kHz sinusoid output. The result is a high frequency output waveform with very low voltage ripple. On the input side, the drive waveform could be generated by a single bidirectional switch that chops the input voltage at the desired transformer frequency. This process generates a high-frequency, square wave input communicated to the ceramic transformer. Due to the bidirectional nature of piezoelectric devices, the resulting design enables high efficiency, typically as high as 98%, which, means that it minimizes power usage and generates very little thermal energy. The result is minimal heat buildup within the transdermal micro-patch 1, which could otherwise require a heat sink to avoid thermal discomfort to the user but at the expense of wearability.

A dual-switch arrangement 36 (or quad pack dual-comparator, if desire to chop both the positive and negative half-cycles) generates an un-rectified output which is communicated to a capacity 37. The capacitors 37 could be composed of tantalum where high efficiency and small size is desired. The capacitor 37 regulates the voltage signal which could then be communicated to a waveform generator 39. An optional quad-diode bridge rectifier 38 could be provided between the capacitor 37 and waveform generator 39 to minimize the levels of the output ripple voltage.

The waveform generator 39 could consist of either a linear or switching bridge amplifier. Since there is generally no need for a step-up ratio, the waveform generator 39 communicates with an amplifier 30 which could include a linear amplifier block, one example being model no. PB50 sold by Apex Microtechnology Corporation located in Tucson, Ariz. The small signal control is preferably generated by the same PROM device 42 as embedded within the comparator switching logic or input side of the waveform generation switch; however, it could also be a separate device if so desired. For example, the small signal control of the waveform generator 39 could be embedded into the overall control architecture facilitating adjustments by a user via a portable electronics graphical user interface (GUI).

In some embodiments, the flextensional transducers as described herein could incorporate a variety of sensor to monitor current (Hall Effect), voltage, frequency, temperature, fluidic pressure, and surface pressure. A feedback control 43 communicates measured data to the PROM device 42 via analog/digital inputs. The internal logic encoded within the PROM device 42 processes this data, thereafter communicating adjustments via controls 51, 52, and/or 53.

As is evident from the explanation above, the described transdermal micro-patch and variations thereof facilitate the oxygenation of living tissue including wounds, the delivery of nutrients and medications to tissues, and the extraction of fluids from tissues. Specific applications include the treatment of longer term illnesses including, but not limited to, cancer, diabetes, and acquired immune deficiency syndrome (AIDS), as well as the treatment of lesions, sores, wounds, and injuries. Accordingly, the described invention is expected to be used by medical practitioners, hospitals, and the like for the treatment of diseases, injuries, and illnesses, as well as medical testing and monitoring.

The description above indicates that a great degree of flexibility is offered in terms of the invention. Although devices and methods have been described in considerable detail with reference to certain preferred versions thereof, other versions are possible. Therefore, the spirit and scope of the appended claims should not be limited to the description of the preferred versions contained herein.

What is claimed is:

1. A transdermal micro-patch for use on a living tissue comprising:
(a) a first membrane being permeable so as to allow passage of a fluid;
(b) a reservoir which stores said fluid;
(c) a micro-pump which communicates said fluid between said reservoir and said first membrane;
(d) at least one flextensional transducer which independently generate ultrasonic waves that are separately communicated into said living tissue and increase the permeability of said living tissue so as to facilitate transport of said fluid between said living tissue and said first membrane; and
(e) a microelectronics circuit which controls functionality of said at least one flextensional transducer and said micro-pump, said reservoir, said micro-pump, said at least one flextensional transducer, and said microelectronics disposed along one side of said first membrane.

2. The transdermal micro-patch of claim 1, further comprising:
(f) an adhesive dispose along said first membrane opposite of said at least one flextensional transducer.

3. The transdermal micro-patch of claim 1, further comprising:
(f) a second membrane with said micro-pump, said reservoir, said at least one flextensional transducer, and said microelectronics circuit disposed between said first membrane and said second membrane.

4. The transdermal micro-patch of claim 1, further comprising:
(f) a matrix disposed about said at least one flextensional transducer.

5. The transdermal micro-patch of claim 1, wherein said transdermal micro-patch delivers said fluid into said living tissue and/or removes said fluid from said living tissue.

6. The transdermal micro-patch of claim 1, wherein at least two of said flextensional transducers communicate separate waves into said living tissue which interact along at least one interaction zone.

7. The transdermal micro-patch of claim 1, further comprising:
(f) a sensor which monitors at least one condition within said transdermal micro-patch or said living tissue so as to facilitate adjustments to the performance of said at least one flextensional transducer and/or said micro-pump when said at least one condition is indicative of damage or irritation to said living tissue.

8. A method of delivering or extracting a fluid between a tissue and a transdermal micro-patch including a reservoir, a micro-pump, at least one flextensional transducer, a membrane, and a microelectronics circuit comprising the steps of:
(a) actuating said micro-pump to communicate said fluid between said reservoir and said membrane;
(b) actuating said at least one flextensional transducer to separately generate ultrasonic waves within said wound area, said ultrasonic waves increase the permeability within said tissue; and
(c) transporting said fluid between said membrane and said tissue, said actuating steps controlled by said microelectronics circuit.

9. The method of claim 8, wherein a large quantity of said fluid is extracted or delivered uninterrupted.

10. The method of claim 8, wherein said micro-pump has a removable cartridge that facilitates continuous transdermal fluidic delivery or extraction without adjustment, removal, or reconfiguration of said reservoir, said micro-pump, said at least one flextensional transducer, said membrane, and/or said microelectronics circuit, said transdermal micro-patch attached to said tissue so that said membrane acts as a barrier until the transdermal fluidic transfer is safe to continue.

11. The method of claim 8, wherein said actuating step is performed at a frequency in the range of 10 to 100 kHz.

12. The method of claim 8, wherein said transporting step moves said fluid from said tissue to said transdermal micro-patch and/or from said transdermal micro-patch to said tissue.

13. The method of claim 8, wherein said actuating step communicates at least two separate waves into said tissue which interact to enhance the performance of said transdermal micro-patch.

14. The method of claim 8, further comprising the steps of:
(d) sensing a condition within said transdermal micro-patch and/or said tissue; and
(e) adjusting the performance of said at least one flextensional transducer and/or said micro-pump when said condition is indicative of damage or irritation to said tissue.

15. The method of claim 14, wherein said condition is flow rate, pressure, temperature, voltage, current, frequency, or amplitude.

16. The method of claim 8, wherein a digital controlled piezo-transformer and a piezoelectric pump mechanism are electrically interconnected in a feedback arrangement so as to enable the highly efficient transfer of said fluid between said tissue and said membrane and between said membrane and said reservoir in a manner that is highly compact and lightweight.
